(19) United States
(12) Patent Application Publication
(10) Pub. No.: US 2025/0082214 A1
(43) Pub. Date: Mar. 13, 2025

(54) WRIST BLOOD PRESSURE MONITOR

(71) Applicant: OMRON HEALTHCARE Co., Ltd., Kyoto (JP)

(72) Inventors: Naomi MATSUMURA, Kyoto (JP); Yukiya SAWANOI, Kyoto (JP)

(21) Appl. No.: 18/955,437

(22) Filed: Nov. 21, 2024

Related U.S. Application Data
(63) Continuation of application No. PCT/JP2023/003177, filed on Feb. 1, 2023.

(30) Foreign Application Priority Data
Jun. 27, 2022 (JP) 2022-102491

(51) Int. Cl. A61B 5/022; A61B 5/00

(52) U.S. Cl. CPC A61B 5/02233; A61B 5/02225; A61B 5/681

(57) ABSTRACT

A wrist blood pressure monitor having a band-like member connected to a main body portion, a sensing cuff, and a pressure cuff, where the cuffs are provided to the band-like member, the band-like member is configured to be wrappable around either a left wrist or a right wrist, the pressing cuff is provided so as to be capable of pressing the sensing cuff toward a left wrist side or a right wrist side when the band-like member is wrapped around the left wrist or the right wrist, the band-like member includes a first end portion and a second end portion that are positioned in a width direction orthogonal to a circumferential direction when the band-like member is wrapped around the left wrist, or right wrist. In some embodiments, the band-like member is wrapped around the right wrist or the left wrist in a first or second state, respectively.

### CROSS-REFERENCE TO RELATED APPLICATIONS

This application is the U.S. national stage application filed pursuant to 35 U.S.C. 365(c) and 120 as a continuation of International Patent Application No. PCT/JP2023/003177, filed Feb. 1, 2023, which application claims priority to Japanese Patent Application No. 2022-102491, filed Jun. 27, 2022, which applications are incorporated herein by reference in their entireties.

### TECHNICAL FIELD

The present disclosure relates to a wrist blood pressure monitor.

### BACKGROUND ART

As a known wrist blood pressure monitor, JP 2006-149682 A (Patent Document 1) discloses a wrist blood pressure monitor including a device body and a cuff band joined to the device body and wrapped around a wrist. A side edge of the cuff band positioned on a fingertip side of a palm when the cuff band is wrapped around the wrist is provided with a band piece protruding toward the fingertip side by a predetermined distance. In the wrist blood pressure monitor disclosed in Patent Document 1, an air bag provided in the cuff band can be arranged at an appropriate position by aligning a distal end of the band piece with a base of the wrist.

### CITATION LIST

### Patent Literature

Patent Document 1: JP 2006-149682 A

### SUMMARY OF INVENTION

### Technical Problem

Given a circumferential surface of the wrist in a circumferential direction being defined as a front surface of an arm positioned on the palm side, a side surface of the arm positioned on a thumb side, a side surface of the arm positioned on a little finger side, and a front surface of the arm positioned on a back side of the hand, the wrist blood pressure monitor disclosed in Patent Document 1 is, when attached to the wrist, disposed with the device body positioned on the front surface of the arm positioned on the palm side and with the air bag provided in the cuff band also positioned covering the front surface of the arm positioned on the palm side. Therefore, when the air bag is inflated, the two arteries of a radial artery running on a base side of the thumb and an ulnar artery running on a base side of the little finger are compressed.

The ulnar artery runs at a position deeper in the arm than the radial artery. Therefore, as the front surface of the arm positioned on the palm side is compressed by a single cuff (air bag), subtle differences occur in a cuff pressure at which the artery starts to collapse and a cuff pressure at which the artery completely collapses between the radial artery and the ulnar artery. This causes a change in the acquired pulse wave amplitude to spread out from a low pressure to a high pressure and, as a result, a peak to appear in two locations, depending on the person. As a result, a measurement error occurs.

As a consideration to improve such measurement errors, it is conceivable to provide a structure for the cuff that includes a pressing cuff and a sensing cuff with independent air systems, allowing the sensing cuff to selectively acquire the pulse wave signal from the radial artery.

However, as disclosed in Patent Document 1, in a case in which the wrist blood pressure monitor is attached to the wrist so that the distal end of the band piece provided on one side in a width direction of the cuff band is aligned with the base of the wrist and the device body of the device is positioned on the front surface of the arm on the palm side, when the sensing cuff is provided so as to match the position of the radial artery of the left hand, the sensing cuff is displaced from the radial artery of the right hand when the wrist blood pressure monitor is attached to the right hand. This makes it difficult to interchangeably use the wrist blood pressure monitor on both the left wrist and the right wrist.

The present disclosure has been made in view of problems such as described above, and an object of the present disclosure is to provide a wrist blood pressure monitor that can be used interchangeably on both a left wrist and a right wrist and can measure blood pressure with high accuracy.

### Solution To Problem

A wrist blood pressure monitor based on an example of the present disclosure includes a main body portion, a band-like member connected to the main body portion, a sensing cuff having a bag-like shape, and a pressing cuff, the sensing cuff and the pressing cuff being provided to the band-like member. The band-like member is configured to be wrappable around either a left wrist or a right wrist. The pressing cuff is provided so as to be capable of pressing the sensing cuff toward a left wrist side or a right wrist side when the band-like member is wrapped around the left wrist or the right wrist. The band-like member includes a first end portion and a second end portion that are positioned in a width direction orthogonal to a circumferential direction when the band-like member is wrapped around the left wrist. In a first state in which the band-like member is wrapped around the left wrist with the first end portion positioned on a finger side of a left hand, the second end portion positioned on an elbow side of the left hand, and the main body portion disposed on a side surface of a left arm on a thumb side of the left hand, the sensing cuff is disposed so as to be capable of compressing a radial artery of the left wrist from a front surface side of the left arm on a palm side. In a second state in which the band-like member is wrapped around the right wrist with the second end portion positioned on a finger side of a right hand, the first end portion positioned on an elbow side of the right hand, and the main body portion disposed on a side surface of a right arm on a thumb side of the right hand, the sensing cuff is disposed so as to be capable of compressing a radial artery of the right wrist from a front surface side of the right arm on a palm side.

In the wrist blood pressure monitor based an example of the present disclosure described above, preferably, in the first state, the sensing cuff is disposed so as to be capable of compressing the radial artery of the left wrist in a portion of a circumferential surface of the left arm from a top portion of the side surface on the thumb side of the left hand to a flexor carpi radialis. Further, preferably, in the second state, the sensing cuff is disposed so as to be capable of compressing a radial artery of the right wrist in a portion of a circumferential surface of the right arm from a top portion of the side surface on the thumb side of the right hand to a flexor carpi radialis.

In the wrist blood pressure monitor based on an example of the present disclosure, the main body portion may include a display unit. In this case, preferably, the display unit includes a first end positioned on the finger side of the left hand and a second end positioned on the elbow side of the left hand in the first state, and the first end of the display unit is positioned on the elbow side of the right hand, and the second end of the display unit is positioned on the finger side of the right hand in the second state.

### Advantageous Effects Of Invention

According to the present disclosure, it is possible to provide a wrist blood pressure monitor that can be used interchangeably on both a left wrist and a right wrist and can measure blood pressure with high accuracy.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is an outer perspective view of a wrist blood pressure monitor according to an embodiment;

FIG. 2 is a schematic cross-sectional view of the wrist blood pressure monitor according to the embodiment;

FIG. 3 is a perspective view illustrating a first state in which the wrist blood pressure monitor according to the embodiment is attached to a left wrist;

FIG. 4 is a schematic cross-sectional view taken along line IV-IV illustrated in FIG. 3;

FIG. 5 is a perspective view illustrating a second state in which the wrist blood pressure monitor according to the embodiment is attached to a right wrist;

FIG. 6 is a cross-sectional view taken along line VI-VI illustrated in FIG. 5;

FIG. 7 is a block diagram illustrating a control configuration of the wrist blood pressure monitor according to the embodiment; and,

FIG. 8 is a flowchart illustrating a measurement flow of the wrist blood pressure monitor according to the embodiment.

### DESCRIPTION OF EMBODIMENTS

Embodiments of the present disclosure will be described in detail below with reference to the drawings. Note that, in the following embodiments, identical or common components are given the same reference numerals in the drawings, and descriptions thereof are not repeated.

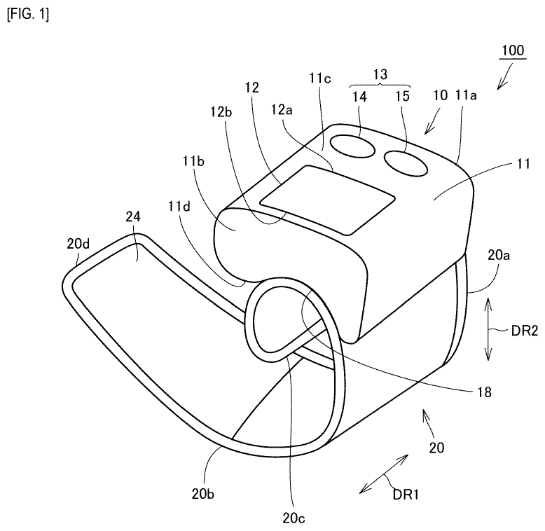

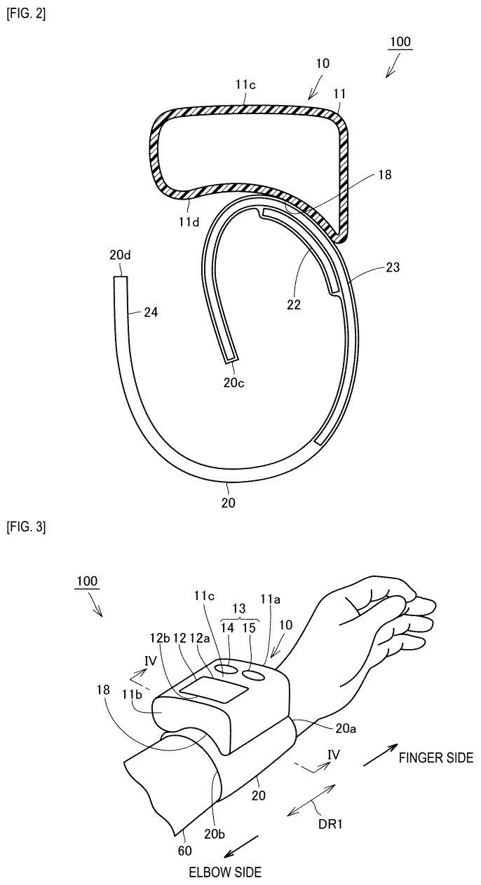

FIG. 1 is an outer perspective view of a wrist blood pressure monitor according to an embodiment. FIG. 2 is a schematic cross-sectional view of the wrist blood pressure monitor according to the embodiment. FIG. 3 is a perspective view illustrating a first state in which the wrist blood pressure monitor according to the embodiment is attached to a left wrist. Note that FIG. 1 illustrates a perspective view in a case in which the wrist blood pressure monitor is viewed from a direction substantially corresponding to that in FIG. 3. A wrist blood pressure monitor 100 according to the embodiment will be described with reference to FIG. 1 to FIG. 3.

As illustrated in FIG. 1 to FIG. 3, the wrist blood pressure monitor 100 according to the embodiment includes a main body portion 10, a band-like member 20, a sensing cuff 22, and a pressing cuff 23.

The band-like member 20 is provided wrappable around either a left wrist or a right wrist. The band-like member 20 includes a first end portion 20a and a second end portion 20b that are positioned in a width direction (direction DR1 in FIG. 1) orthogonal to a circumferential direction in a state of being wrapped around the left wrist. In a case in which the band-like member 20 is wrapped around the left wrist, the band-like member 20 is wrapped so that the first end portion 20a is positioned on a finger side of the left hand and the second end portion 20b is positioned on an elbow side of the left hand.

The band-like member 20 includes an outer cover member positioned radially outward when in a wrapped state, and an inner cover member positioned radially inward and in contact with a circumferential surface of the wrist when in the wrapped state.

The band-like member 20 is formed in a bag-like shape by the outer cover member and the inner cover member being layered on one another, with peripheral edges thereof being covered by a bias tape and joined (by sewing or welding, for example).

A surface fastener (not illustrated) is provided on an outer circumferential surface of the band-like member 20 at/near a first end portion 20c in a longitudinal direction (circumferential direction in the wrapped state), and a surface fastener 24 is provided on an inner circumferential surface of the band-like member 20 at/near a second end portion 20d positioned on a side opposite to the first end portion 20c. The surface fastener on the first end portion 20c side is constituted by a hook fastener, for example, and the surface fastener 24 on the second end portion 20d side is constituted by a loop fastener, for example.

The band-like member 20 is provided with the sensing cuff 22 and the pressing cuff 23. The sensing cuff 22 and the pressing cuff 23 are disposed between the outer cover member and the inner cover member constituting the band-like member 20.

The pressing cuff 23 is provided so as to be capable of pressing the sensing cuff 22 toward the wrist. Specifically, the pressing cuff 23 is provided covering the sensing cuff 22 from the side opposite to the side where the wrist is positioned in the wrapped state.

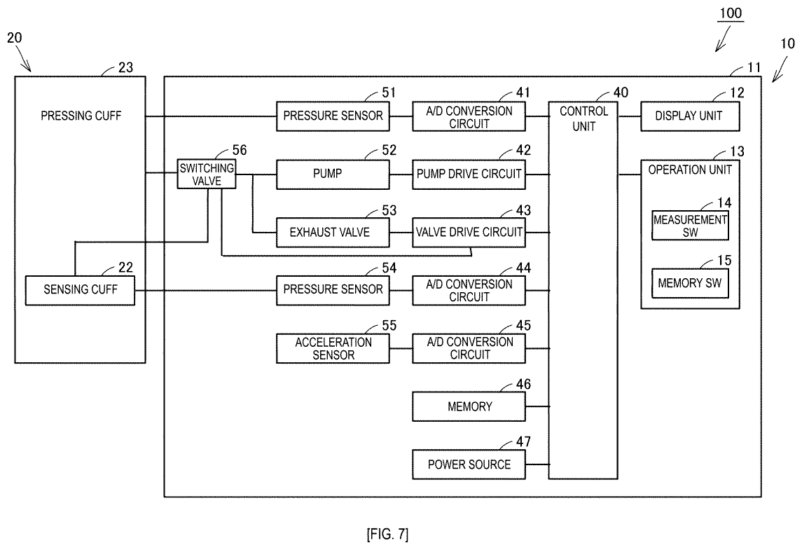

The sensing cuff 22 and the pressing cuff 23 are provided so as to be capable of expanding and contracting as fluid enters and exits. The sensing cuff 22 and the pressing cuff 23 are connected to a pump 52 (See FIG. 7) accommodated in the case 11. The sensing cuff 22 and the pressing cuff 23 are inflated by air being introduced as a fluid by the pump 52.

Note that, in the above description, a case in which the band-like member 20 is formed into a bag-like shape by the outer cover member and the inner cover member has been described as an example. However, in a case in which the band-like member 20 is constituted by a belt member or the like, the sensing cuff 22 and the pressing cuff 23 may be provided on an inner front surface side of the band-like member 20 positioned radially inward in the wrapped state.

The main body portion 10 includes a case 11 having a substantially box shape. The case 11 includes a first side surface 11a, a second side surface 11b, an upper surface 11c, and a bottom surface 11d. Note that, as described below, a circuit board on which various electronic components are mounted, the pump 52 (See FIG. 7), and the like are accommodated in the case 11.

The first side surface 11a is positioned on the first end portion 20a side of the band-like member 20. The first side surface 11a connects end portions of the upper surface 11c and the bottom surface 11d positioned on the first end portion 20a side. The second side surface 11b is positioned on the second end portion 20b side of the band-like member 20. The second side surface 11b connects end portions of the upper surface 11c and the bottom surface 11d positioned on the second end portion 20b side.

The upper surface 11c and the bottom surface 11d face each other in a vertical direction (e.g., direction DR2 in FIG. 1). A display unit 12 and an operation unit 13 are provided on the upper surface 11c. The display unit 12 and the operation unit 13 are disposed side by side in a direction parallel to the width direction described above. The display unit 12 is positioned on the second end portion 20b side of the band-like member 20. That is, the display unit 12 is positioned on the second side surface 11b side. The operation unit 13 is positioned on the first end portion 20a side of the band-like member 20. That is, the operation unit 13 is positioned on the first side surface 11a side. The bottom surface 11d is provided with a curved portion 18 conforming to a side surface of an arm on a thumb side.

The display unit 12 includes a first end 12a and a second end 12b. The first end 12a is positioned on the first end portion 20a side of the band-like member 20, and the second end 12b is positioned on the second end portion 20b side of the band-like member 20. That is, the first end 12a is positioned on the first side surface 11a side, and the second end 12b is positioned on the second side surface 11b side.

The display unit 12 displays information relating to blood pressure measurement such as blood pressure measurement results and other information. Note that the display unit 12 is constituted by a display such as an organic electroluminescent (hereinafter “EL”) display or a liquid crystal display. The operation unit 13 includes, for example, a measurement switch 14 and a memory switch 15. The measurement switch 14 and the memory switch 15 are disposed side by side.

The measurement switch 14 is a button pressed when measurement is to be started. The memory switch 15 is a button pressed when a measured value or the like is to be stored or when a stored measurement value or the like is to be displayed.

Note that the operation unit 13 is not limited to being a push type switch and may be, for example, a pressure sensitive type (resistance type) or a proximity type (capacitance type) touch panel type switch. In this case, the switch is displayed on the display unit 12. Further, a microphone (not illustrated) may be provided for input of a start blood pressure measurement instruction from a user via sound. The operation unit 13 may be provided with a power switch.

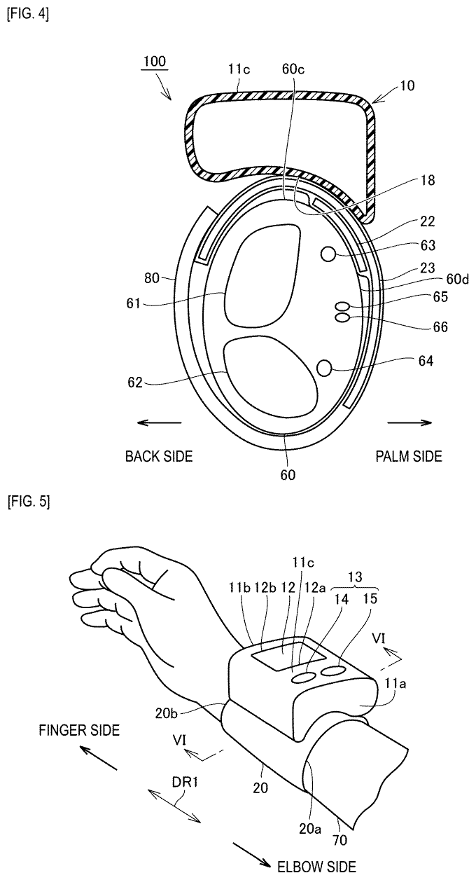

FIG. 4 is a schematic cross-sectional view taken along line IV-IV illustrated in FIG. 3. The state of the wrist blood pressure monitor 100 wrapped around the left wrist will now be described with reference to FIG. 3 and FIG. 4.

As illustrated in FIG. 3, in a case in which blood pressure is to be measured on the left wrist side, the band-like member 20 is wrapped around the left wrist, positioning the first end portion 20a of the band-like member 20 on the finger side of the left hand, positioning the second end portion 20b on the elbow side of the left hand, and disposing the main body portion 10 on a side surface 60c of a left arm 60 on the thumb side of the left hand (See FIG. 4). The main body portion 10 is positioned relative to the left wrist by the curved portion 18 described above, and is disposed conforming to the side surface 60c of the left arm 60.

In a first state in which the band-like member 20 is thus wrapped around the left wrist, the first end 12a of the display unit 12 is positioned on the finger side of the left hand, and the second end 12b of the display unit 12 is positioned on the elbow side of the left hand. Further, on the upper surface 11c, the display unit 12 is positioned on the elbow side of the left hand, and the operation unit 13 is positioned on the finger side of the left hand.

The user can confirm that the wrist blood pressure monitor 100 is appropriately attached to the left wrist by confirming a positional relationship between the first end portion 20a and the second end portion 20b of the band-like member 20, a positional relationship between the first end 12a and the second end 12b of the display unit 12, or a positional relationship between the display unit 12 and the operation unit 13.

Note that, as a method for confirming that the wrist blood pressure monitor 100 is appropriately attached to the left wrist, a mark indicating positioning on a side opposite to the finger side (elbow side) when the wrist blood pressure monitor 100 is attached to the left wrist may be provided on a portion positioned on the second end portion 20b side of the band-like member 20, the second side surface 11b of the case 11, or a portion positioned on the second side surface 11b side of the upper surface 11c.

Further, a mark indicating positioning on the finger side when the wrist blood pressure monitor 100 is attached to the left wrist may be provided on a portion positioned on the first end portion 20a side of the band-like member 20, the first surface side 11a of the case 11, or a portion positioned on the first side surface 11a side of the upper surface 11c.

As illustrated in FIG. 4, a radius 61, an ulna 62, a radial artery 63, an ulnar artery 64, a flexor carpi radialis 65, and a palmaris longus 66 run inside the left arm 60 on the left wrist side. In a case in which the position of the left hand is adjusted so that the thumb of the hand faces upward, the radius 61 and the ulna 62 are aligned in the vertical direction, and the radius 61 is positioned upward of the ulna 62. Further, the radial artery 63 is positioned on the palm side with respect to the radius 61, and the ulnar artery 64 is positioned on the palm side with respect to the ulna 62. The flexor carpi radialis 65 and the palmaris longus 66 are disposed between the radial artery 63 and the ulnar artery 64.

In the first state in which the band-like member 20 is wrapped around the left wrist as described above, the sensing cuff 22 is disposed so as to be capable of compressing the radial artery 63 of the left wrist from a front surface 60d side of the left arm on the palm side. More specifically, the sensing cuff 22 is disposed on the left wrist so as to be capable of compressing the radial artery 63 of the left wrist in a portion of a circumferential surface of the left arm 60 from a top portion of the side surface 60c on the thumb side of the left hand to the flexor carpi radialis 65. Accordingly, it is possible to stably compress the radial artery 63 by the sensing cuff 22 while reducing the influence of the radius 61 and the flexor carpi radialis 65.

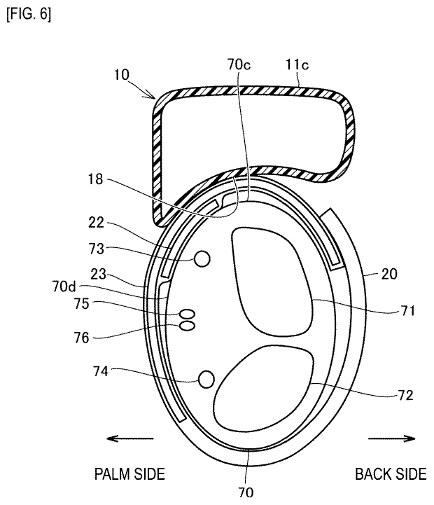

FIG. 5 is a perspective view illustrating a state in which the wrist blood pressure monitor according to the embodiment is attached to the right wrist. FIG. 6 is a cross-sectional view taken along line VI-VI illustrated in FIG. 5.

As illustrated in FIG. 5, in a case in which blood pressure is to be measured on the right wrist side, the band-like member 20 is wrapped around the right wrist, positioning the second end portion 20b of the band-like member 20 on a finger side of the right hand, positioning the first end portion 20a on an elbow side of the right hand, and disposing the main body portion 10 on a side surface 70c of a right arm 70 on the thumb side of the right hand (See FIG. 6). The main body portion 10 is positioned relative to the right wrist by the curved portion 18 described above, and is disposed conforming to the side surface 70c of the right arm 70.

In a second state in which the band-like member 20 is thus wrapped around the right wrist, the first end 12a of the display unit 12 is positioned on the elbow side of the right hand, and the second end 12b of the display unit 12 is positioned on the finger side of the right hand. Further, on the upper surface 11c, the display unit 12 is positioned on the finger side of the right hand, and the operation unit 13 is positioned on the elbow side of the right hand.

The user can confirm that the wrist blood pressure monitor 100 is appropriately attached to the right wrist by confirming a positional relationship between the first end portion 20a and the second end portion 20b of the band-like member 20, a positional relationship between the first end 12a and the second end 12b of the display unit 12, or a positional relationship between the display unit 12 and the operation unit 13.

Note that, as a method for confirming that the wrist blood pressure monitor 100 is appropriately attached to the right wrist, a mark indicating positioning on a side opposite to the finger side (elbow side) when the wrist blood pressure monitor 100 is attached to the right wrist may be provided on a portion positioned on the first end portion 20a side of the band-like member 20, the first side surface 11a of the case 11, or a portion positioned on the first side surface 11a side of the upper surface 11c.

Further, a mark indicating positioning on the finger side when the wrist blood pressure monitor 100 is attached to the right wrist may be provided on a portion positioned on the second end portion 20b side of the band-like member 20, the second side surface 11b of the case 11, or a portion positioned on the second side surface 11b side of the upper surface 11c.

As illustrated in FIG. 6, a radius 71, an ulna 72, a radial artery 73, an ulnar artery 74, a flexor carpi radialis 75, and a palmaris longus 76 run inside the right arm 70 on the right wrist side. In a case in which the position of the hand is adjusted so that the thumb of the right hand faces upward, the radius 71 and the ulna 72 are aligned in the vertical direction, and the radius 71 is positioned upward of the ulna 72. Further, the radial artery 73 is positioned on the palm side with respect to the radius 71, and the ulnar artery 74 is positioned on the palm side with respect to the ulna 72. The flexor carpi radialis 75 and the palmaris longus 76 are disposed between the radial artery 73 and the ulnar artery 74.

In the second state in which the band-like member 20 is wrapped around the right wrist as described above, the sensing cuff 22 is disposed so as to be capable of compressing the radial artery 73 of the right wrist from a front surface 70d side of the right arm on the palm side. More specifically, the sensing cuff 22 is disposed on the right wrist so as to be capable of compressing the radial artery 73 of the right wrist in a portion of a circumferential surface of the right arm 70 from a top portion of the side surface 70c on the thumb side of the right hand to the flexor carpi radialis 75. Accordingly, it is possible to stably compress the radial artery 73 by the sensing cuff 22 while reducing the influence of the radius 71 and the flexor carpi radialis 75.

FIG. 7 is a block diagram illustrating a control configuration of the wrist blood pressure monitor according to the embodiment. Functional blocks of the wrist blood pressure monitor will be described with reference to FIG. 7.

As illustrated in FIG. 7, the main body portion 10 includes, in addition to the display unit 12 and the operation unit 13 described above, a control unit 40, an A/D conversion circuit 41, a pump drive circuit 42, a valve drive circuit 43, A/D conversion circuits 44, 45, a memory 46, a power source 47, a pressure sensor 51, the pump 52, an exhaust valve 53, a pressure sensor 54, an acceleration sensor 55, and a switching valve 56. These components are accommodated in the case 11.

The power source 47 supplies power to the various elements mounted in the main body portion 10. The power source 47 is, for example, a rechargeable secondary battery.

The memory 46 non-transitorily stores various data of a program for controlling the wrist blood pressure monitor 100 and the like, settings data for setting various functions of the wrist blood pressure monitor 100, data of measurement results of blood pressure values, and the like. Further, the memory 46 is used as working memory and the like for executing a program.

The control unit 40 executes various functions in accordance with the program for controlling the wrist blood pressure monitor 100 stored in the memory 46. The control unit 40 can execute blood pressure measurement based on an oscillometric method.

The control unit 40 drives the pump drive circuit 42 to control the operation of the pump 52. Further, the control unit 40 drives the valve drive circuit 43 to control the operation of the exhaust valve 53 and the switching valve 56. The pump 52, the exhaust valve 53, and the switching valve 56 are connected to the sensing cuff 22 and the pressing cuff 23 via piping.

The pump 52 is constituted by a piezoelectric pump, for example. The switching valve 56 switches a connection destination of the pump 52 and the exhaust valve 53 to the sensing cuff 22 or the pressing cuff 23. The exhaust valve 53 is openably and closeably provided and controls an open/closed state of the exhaust valve 53 and the switching valve 56, making it possible to control a state in which air in the sensing cuff 22 and the pressing cuff 23 can be exhausted.

The pressure sensor 51 (first pressure sensor) detects the pressure in the pressing cuff 23. The pressure sensor 51 is, for example, a piezoresistive pressure sensor. Pressure data detected by the pressure sensor 51 is converted into predetermined data by the A/D conversion circuit 41 and input to the control unit 40.

The pressure sensor 54 (second pressure sensor) detects the pressure in the sensing cuff 22. The pressure sensor 54 is, for example, a piezoresistive pressure sensor. Pressure data detected by the pressure sensor 54 is converted into predetermined data by the A/D conversion circuit 44 and input to the control unit 40.

The acceleration sensor 55 is, for example, a 3-axis acceleration sensor. The acceleration sensor 55 detects acceleration information representing acceleration in three directions orthogonal to one another. The acceleration information detected by the acceleration sensor 55 is converted into predetermined data by the A/D conversion circuit 45 and input to the control unit 40.

The acceleration information is an example of information representing movement of a subject. The control unit 40 can calculate not only walking of the subject, but also an amount of activity in various activities such as household chores and desk work by using the acceleration information. The amount of activity is, for example, an indicator associated with the activity of the subject, such as a movement (walking) distance, calorie consumption, or an amount of fat burned. The control unit 40 can also conduct estimations of the subject before sleeping, during sleeping, and after waking by using the acceleration information. Further, the control unit 40 can also estimate whether the subject is exercising by using the acceleration information.

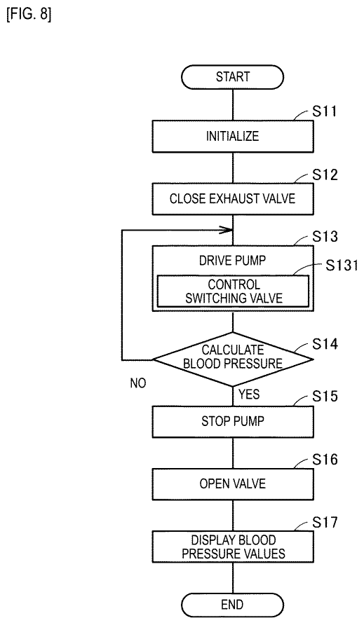

FIG. 8 is a flowchart illustrating a measurement flow of the wrist blood pressure monitor according to the embodiment. With reference to FIG. 8, the measurement flow of the wrist blood pressure monitor is detailed below.

When blood pressure measurement is started with the wrist blood pressure monitor 100 attached to the left wrist or the right wrist, initialization is first implemented in step (S11). Note that the initialization is implemented on the basis of detection of a measurement instruction, such as the measurement switch 14 being pressed. Specifically, in step (S11), the control unit 40 initializes a processing memory region of the memory 46. Further, in step (S11), the control unit 40 controls the pump drive circuit 42 to turn off the pump 52, and controls the valve drive circuit 43 to open the exhaust valve 53 while adjusting the switching valve 56, thereby exhausting the air in the pressing cuff 23 and the sensing cuff 22. Thus, the pressing sensors 51, 54 are adjusted to 0 mmHg, and the pressing sensors 51, 54 are initialized.

Next, in step (S12), the exhaust valve 53 is closed. Specifically, the control unit 40 brings the exhaust valve 53 to a closed state via the valve drive circuit 43.

Subsequently, in step (S13), the control unit 40 controls the pump drive circuit 42 to drive the pump 52. Step (S13) includes step (S131) of controlling the switching valve 56. In step (S131), first, the control unit 40 controls the valve drive circuit 43 to control the state of the switching valve 56 so that the fluid can be supplied to the sensing cuff 22. As a result, air is supplied as a fluid from the pump 52 to the sensing cuff 22, thereby pressurizing the sensing cuff 22.

At this time, the control unit 40 determines whether the fluid supplied to the sensing cuff 22 has reached an intended supply amount. For example, the control unit 40 determines from the output of the pressure sensors 51, 54 whether the pressure of the sensing cuff 22 has reached a predetermined pressure (for example, 15 mmHg) or whether a drive time of the pump 52 has exceeded a predetermined time.

When the control unit 40 determines that the pressure of the sensing cuff 22 has not reached the predetermined pressure or the drive time of the pump 52 has not exceeded the predetermined time, pressurization is continuously implemented.

When the control unit 40 determines that the predetermined pressure has been reached or the predetermined time has been exceeded, the control unit 40, in step (S131), controls the valve drive circuit 43 to control the switching valve 56 so that the amount of fluid in the sensing cuff 22 is kept constant. Specifically, the control unit 40 controls the state of the switching valve 56 so that the fluid can be supplied to the pressing cuff 23. As a result, air is supplied from the pump 52 to the pressing cuff 23, thereby pressurizing the pressing cuff 23.

Accordingly, the pressure of the pressing cuff 23 gradually increases. By a pressing force that occurs with this rise in pressure, the pressing cuff 23 presses the sensing cuff 22 toward the left wrist or the right wrist, and the sensing cuff 22 compresses the radial artery. In this pressurization process, the control unit 40 detects the pressure of the sensing cuff 22 by the pressure sensor 54 and acquires a pulse wave signal as a variable component.

Next, in step (S14), the control unit 40 calculates blood pressure values (systolic blood pressure, “SBP,” and diastolic blood pressure, “DBP”) on the basis of the acquired pulse wave signal. As a method for calculating the blood pressure values, known algorithms based on the oscillometric method can be used.

At this time, the control unit 40 determines whether the blood pressure values have been measured. When the control unit 40 determines that the blood pressure values have not been measured (step (S14): NO), the processing returns to step (S13) and the pressing cuff 23 is pressurized unless the pressing cuff 23 has reached an upper limit (for example, 300 mmHg, which is a predetermined value for safety).

On the other hand, when the control unit 40 determines that the blood pressure values have been measured (step (S14): YES), step (S15) is implemented. In step (S15), the control unit 40 controls the pump drive circuit 42 to stop the pump 52.

Subsequently, in step (S16), the control unit 40 controls the valve drive circuit 43 to bring the exhaust valve 53 to an open state while adjusting the switching valve 56. As a result, the fluid in the pressing cuff 23 and the sensing cuff 22 is discharged. Next, in step (S17), the measurement results of the blood pressure values are displayed on the display unit 12. The blood pressure can be measured through such steps (S11) to (S17).

As described above, in the wrist blood pressure monitor 100 according to the embodiment, in a case in which the blood pressure is to be measured on the left wrist side, the band-like member 20 is wrapped around the left wrist, positioning the first end portion 20a on the finger side of the left hand, positioning the second end portion 20b on the elbow side of the left hand, and disposing the main body portion 10 on the side surface of the left arm on the thumb side of the left hand. In such a state, the sensing cuff 22 is disposed so as to be capable of compressing the radial artery 63 of the left wrist from the front surface 60d side of the left arm 60 on the palm side.

Further, in a case in which blood pressure is to be measured on the right wrist side, the band-like member 20 is wrapped around the right wrist, positioning the second end portion 20b on the finger side of the right hand, positioning the first end portion 20a on the elbow side of the right hand, and disposing the main body portion 10 on the side surface of the right arm on the thumb side of the right hand. In such a state, the sensing cuff 22 is disposed so as to be capable of compressing the radial artery 73 of the right wrist from the front surface 70d side of the right arm 70 on the palm side.

Therefore, whether the band-like member 20 is wrapped around the left wrist or the right wrist, the radial artery of the left wrist or the right wrist can be compressed by the sensing cuff 22. As a result, the wrist blood pressure monitor 100 can be used interchangeably on both the left wrist and the right wrist and can measure blood pressure with high accuracy.

### SUPPLEMENTARY NOTES

As described above, the present embodiment includes the following disclosures.

### Configuration 1

A wrist blood pressure monitor comprising:
a main body portion;
a band-like member connected to the main body portion;
a sensing cuff having a bag-like shape; and
a pressing cuff, wherein
the sensing cuff and the pressing cuff are provided to the band-like member,
the band-like member is configured to be wrappable around either a left wrist or a right wrist,
the pressing cuff is provided so as to be capable of pressing the sensing cuff toward a left wrist side or a right wrist side when the band-like member is wrapped around the left wrist or the right wrist,
the band-like member includes a first end portion and a second end portion that are positioned in a width direction orthogonal to a circumferential direction when the band-like member is wrapped around the left wrist,
in a first state in which the band-like member is wrapped around the left wrist with the first end portion positioned on a finger side of a left hand, the second end portion positioned on an elbow side of the left hand, and the main body portion disposed on a side surface of a left arm on a thumb side of the left hand, the sensing cuff is disposed so as to be capable of compressing a radial artery of the left wrist from a front surface side of the left arm on a palm side, and
in a second state in which the band-like member is wrapped around the right wrist with the second end portion positioned on a finger side of a right hand, the first end portion positioned on an elbow side of the right hand, and the main body portion disposed on a side surface of a right arm on a thumb side of the right hand, the sensing cuff is disposed so as to be capable of compressing a radial artery of the right wrist from a front surface side of the right arm on a palm side.

### Configuration 2

The wrist blood pressure monitor according to configuration 1, wherein
in the first state, the sensing cuff is disposed so as to be capable of compressing the radial artery of the left wrist in a portion of a circumferential surface of the left arm from a top portion of the side surface on the thumb side of the left hand to a flexor carpi radialis, and
in the second state, the sensing cuff is disposed so as to be capable of compressing a radial artery of the right wrist in a portion of a circumferential surface of the right arm from a top portion of the side surface on the thumb side of the right hand to a flexor carpi radialis.

### Configuration 3

The wrist blood pressure monitor according to configuration 1 or 2, wherein
the main body portion includes a display unit, and
the display unit includes a first end positioned on the finger side of the left hand and a second end positioned on the elbow side of the left hand in the first state, and
the first end of the display unit is positioned on the elbow side of the right hand, and the second end of the display unit is positioned on the finger side of the right hand in the second state.

The embodiments disclosed herein are illustrative in all respects and are not intended as limitations. The scope of the present invention is indicated by the claims and includes all meaning equivalent to the claims and changes within the scope of the claims.

### REFERENCE NUMERALS LIST

10 Main body portion, 11 Case, 11a First side surface, 11b Second side surface, 11c Upper surface, 11d Bottom surface, 12 Display unit, 12a First end, 12b Second end, 13 Operation unit, 14 Measurement switch, 15 Memory switch, 18 Curved portion, 20 Band-like member, 20a First end portion, 20b Second end portion, 20c First end portion, 20d Second end portion, 22 Sensing cuff, 23 Pressing cuff, 24 Surface fastener, 40 Control unit, 41 A/D conversion circuit, 42, Pump drive circuit, 43 Valve drive circuit, 44, 45 A/D conversion circuit, 46 Memory, 47 Power source, 51 Pressure sensor, 52 Pump, 53 Exhaust valve, 54 Pressure sensor, 55 Acceleration sensor, 56 Switching valve, 60 Left arm, 60c Side surface, 60d Front surface, 61 Radius, 62 Ulna, 63 Radial artery, 64 Ulnar artery, 65 Flexor carpi radialis, 66 Palmaris longus, 70 Right arm, 70c Side surface, 70d Front surface, 71 Radius, 72 Ulna, 73 Radial artery, 74 Ulnar artery, 75 Flexor carpi radialis, 76 Palmaris longus, 100 Wrist blood pressure monitor.

What is claimed is:

1. A wrist blood pressure monitor comprising:
a main body portion;
a band-like member connected to the main body portion;
a sensing cuff having a bag-like shape; and
a pressure cuff, wherein
the sensing cuff and the pressure cuff are provided to the band-like member,
the band-like member is configured to be wrappable around either a left wrist or a right wrist,
the pressing cuff is provided so as to be capable of pressing the sensing cuff toward a left wrist side or a right wrist side when the band-like member is wrapped around the left wrist or the right wrist,
the band-like member includes a first end portion and a second end portion that are positioned in a width direction orthogonal to a circumferential direction when the band-like member is wrapped around the left wrist,
in a first state in which the band-like member is wrapped around the left wrist with the first end portion positioned on a finger side of a left hand, the second end portion positioned on an elbow side of the left hand, and the main body portion disposed on a side surface of a left arm on a thumb side of the left hand, the sensing cuff is disposed so as to be capable of compressing a radial artery of the left wrist from a front surface side of the left arm on a palm side, and
in a second state in which the band-like member is wrapped around the right wrist with the second end portion positioned on a finger side of a right hand, the first end portion positioned on an elbow side of the right hand, and the main body portion disposed on a side surface of a right arm on a thumb side of the right hand, the sensing cuff is disposed so as to be capable of compressing a radial artery of the right wrist from a front surface side of the right arm on a palm side.

2. The wrist blood pressure monitor according to claim 1, wherein
in the first state, the sensing cuff is disposed so as to be capable of compressing the radial artery of the left wrist in a portion of a circumferential surface of the left arm from a top portion of the side surface on the thumb side of the left hand to a flexor carpi radialis, and
in the second state, the sensing cuff is disposed so as to be capable of compressing a radial artery of the right wrist in a portion of a circumferential surface of the right arm from a top portion of the side surface on the thumb side of the right hand to a flexor carpi radialis.

3. The wrist blood pressure monitor according to claim 1, wherein
the main body portion includes a display unit, and
the display unit includes a first end positioned on the finger side of the left hand and a second end positioned on the elbow side of the left hand in the first state, and
the first end of the display unit is positioned on the elbow side of the right hand, and the second end of the display unit is positioned on the finger side of the right hand in the second state.

4. The wrist blood pressure monitor according to claim 2, wherein
the main body portion includes a display unit, and
the display unit includes a first end positioned on the finger side of the left hand and a second end positioned on the elbow side of the left hand in the first state, and
the first end of the display unit is positioned on the elbow side of the right hand, and the second end of the display unit is positioned on the finger side of the right hand in the second state.
